(19) United States
(12) Patent Application Publication
(10) Pub. No.: US 2020/0157577 A1
(43) Pub. Date: May 21, 2020

(54) Method for decomposition of the metallorganic matter of graptolite-argillite by microbial consortium

(71) Applicants: Anne MENERT, Tartu (EE); Maia KIVISAAR, Tartu (EE); Sirli SIPPKULLI, Tabasalu, Harku vald (EE); Ain HEINARU, Tartu (EE); Tlit MAIDRE, Tallinn (EE)

(72) Inventors: Anne MENERT, Tartu (EE); Maia KIVISAAR, Tartu (EE); Sirli SIPPKULLI, Tabasalu, Harku vald (EE); Ain HEINARU, Tartu (EE); Tlit MAIDRE, Tallinn (EE)

(73) Assignee: BiotaTec OÜ, Tallinn (EE)

(21) Appl. No.: 15/998,841

(22) PCT Filed: Feb. 16, 2017
(86) PCT No.: PCT/EE2017/000001

§ 371 Date: Sep. 16, 2018

(30) Foreign Application Priority Data
Feb. 16, 2016 (EE) P201600003

(51) Int. Cl. C12P 5/02; C12N 1/20; C22B 3/18; C22B 3/00

(52) U.S. Cl. CPC C12P 5/023; C22B 23/0407; C22B 3/18; C12N 1/20

(57) ABSTRACT

The present invention describes a method, which consists in decomposition of graptolite-argillite organometallic matter in anaerobic environment by a stable adapted microbial consortium, accompanied by bioleaching of metals and methane generation. Supporting experimental data are presented and the effect of betaine in biodegradation of argillite organometallic compounds is demonstrated. Microbial communities provoking these processes are characterized.

### FIELD OF THE INVENTION

The present invention belongs to the field of biotechnology, bioremediation and biohydrometallurgy. The invention describes a method for decomposition of organometallic matter contained in argillite ore by the use of microbial consortium, accompanied by bioleaching of metals and evolution of methane, and suitable environmental conditions and growth media for those processes. The biodegradation capability of microbial community isolated from argillite can be used to eliminate the adverse environmental impact of argillite and to produce useful products emerging in this process.

### BACKGROUND OF THE INVENTION

Relatively deep-lying and low-maturity shales are known to be origins of biogenic methane generation. Methane is formed from the organic part of shale-kerogen. The bore holes drilled into such minerals suffer from low productivity, which can be enhanced by biological methods [WO 2006/118569 A1; U.S. Pat. No. 8,302,683; Patent application WO2008/041990; Patent application CA2801558 A1]. Estonian black shale (graptolite-argillite) consists essentially of organic matter (kerogen) with feldspar, quartz, clay minerals, a small amount of Fe-sulfides and gypsum [Maremäe, 1988]. Kerogen is very difficult to study because it is practically insoluble in most organic solvents, [Aloe, et al., 2006].

Phosphorite is Estonian natural resource with the largest reserve in Europe [Reinsalu, 2012]. Its safe mining, however, is related to the usage opportunities and technologies of layers aligned with this deposit—oil shale and argillite. Primarily, the problem is in graptolite-argillite. Graptolite-argillite is a particular type of oil shale, a hardened clay mineral mixed with organic matter, the resources of which in Estonia are 60 billion tonnes [Bauert, Kattai, 1997]. Because of low content of organic matter (12-17%; calorific value 1500-1600 kcal/kg, or 5-7 MJ/kg), its direct use as a fuel is not possible. Graptolite-argillite contains 2-6% of scattered colonies of ferrous sulfidic mineral-pyrite (FeS₂). Its environmental hazard consists in interaction of pyrite, organic matter, water and oxygen with bacteria. Namely, pyrite reacts with oxygen to generate heat. Iron and sulfur bacteria are activated, being active up to 50-60° C., followed by active oxidation of organic matter (argillite self-ignition) and the temperature increase from 1000 to 1500° C. One of the reaction products is sulfuric acid with the release of toxic gases [Puura et al., 1999]. In the processing of argillite it is therefore necessary to limit the access to oxygen.

Estonian argillite contains significant quantities of heavy metals [Lippmaa et al., 2009], being enriched with uranium (minimum enrichment value, m.e.v. 30 ppm), molybdenum (m.e.v. 200 ppm), vanadium (m.e.v. 1000 ppm), lead (m.e.v. 100 ppm) and cobalt (30 ppm m.e.v.), as well as zinc, rhenium, nickel and other elements [Petersell, 2008; Voolma et al., 2013]. Metals are in argillite as sulfide minerals or in the composition of organometallic compounds (geopolymers). Traditionally, metals have been leached from argillite with acids, by oxidation or hydrogenation [Lippmaa et al., 2011]. In this case organic compounds contained in ores and bound to metals are a major problem. In the years 1949-1952 at Sillamäe over 69 tons of uranium compounds were produced from 250,000 tons of argillite [Aloe, et al., 2006]. Microbial degradation of organometallic complexes and bioleaching of metals would allow to valorize argillite as an environmentally harmful byproduct accompanying phosphorite mining. Corresponding studies in the literature, however, are still missing.

Microbial degradation of geopolymers with methane gas formation has been stimulated with various methanogenic substrates [Meslé et al., 2013; Urios et al., 2012, 2013; Jones et al., 2008; Harris et al., 2008, U.S. Pat. Nos. 9,004,162 B2, 7,696,132], including using methanol and trimethylamine [Wuchter et al., 2013; Patent application WO2009/140313; US patent application 20130116126 A1], but there are no references on the use of betaine for this purpose. Recently, however, betaine (trimethylglycine) consuming methanogens have been described [Watkins, et al., 2014; Ticak et al., 2015]. The role of betaine might be propagation of methanogenesis through providing additional substrate for methylotrophic methanogens [Asakawa et al., 1998; Ticak et al., 2015].

### SUMMARY OF THE INVENTION

The present invention describes a method, which consists in decomposition of organometallic matter of graptolite-argillite by a stable adapted microbial community under anaerobic conditions, which is accompanied by bioleaching of metals and release of methane.

First, the most efficient cultivation medium promoting the degradation of argillite organic matter (kerogen) was selected. When cultivating methanogens in mixed culture the buffering capacity of the medium is of utmost importance because metabolites from fermentative microorganisms acidify the environment rapidly while methanogens prefer solely alkaline region (pH 6.8-7.5). Availability of microelements and vitamins is important; supplement of metabolic intermediates and methanogenic substrates also facilitates the growth of methanogens. Thus, for enrichment of a microbial consortium decomposing organometallic complexes in argillite, a liquid cultivation medium suitable to use is R2A (yeast extract 0.5 g/L; Difco peptone 0.5 g/L Casamino acids 0.5 g/L, glucose 0.5 g/l, soluble starch 0.5 g/L, K₂HPO₄ 0.3 g/L, MgSO₄.7H₂O 0.05 g/L, Na-pyruvate 0.3 g/L), supplemented with betaine, and cultivation in anaerobic batch reactor (under argon atmosphere) at a temperature of 37 [symbol] ° C. The initial pH of culture medium 7.0-7.5 should be maintained until the end of cultivation. If with the medium selected a microbial consortium efficiently using the organic matter of argillite has been obtained, generation of methane into the gas phase (measured by gas chromatograph) refers to it. Using the microbial consortium initially isolated as the inoculum for cultivating fresh argillite samples in liquid R2A medium, with selection and adaptation, a new consortium with better biodegrading ability can be obtained from this consortium, achieving a higher methane yield, and also a better metals bioleaching capability.

Methane release into the gas phase is one evidence of organometallic complexes degradation. The microbial methane yield from argillite might be 10 . . . 250 μmol CH₄/g mineral [Wuchter et al., 2013; Meslé et al., 2015]. If methane yield released into the gas phase is higher, it is an indication that the consortium enriched is an effective organometallic complexes degrader. The origin of methane from the organic part of argillite is tested by isotopic analysis with the δ¹³C method. The ratio of stable isotopes is determined relative to the standard:

$${δ}^{13} C = ( \frac{{( \frac{{13}_{C}}{{12}_{C}} )}_{sample}}{{( \frac{{13}_{C}}{{12}_{C}} )}_{standard}} - 1 ) \times 1000 \%$$

For presenting the results of carbon analysis of carbonate rocks and sediments, the V-PDB (Vienna Belemnitella Americana, Peedee Formation, Cretaceous Period, South Carolina) scale is used, where fossil carbonate is taken as zero-point. δ¹³C (‰ V-PDB) characterizes the difference of stable isotopes ¹³C and ¹²C per thousand units (per mil, ‰), with a positive result indicating that the sample is saturated with the heavier isotope as compared to the standard, and a negative value that the sample is impoverished from the heavier isotope as compared to the standard [Sepp, 2013]. The typical values by δ¹³C (‰ V-PDB) for methane originating from kerogen material are of −50 . . . −70‰.

Another evidence for degradation of organometallic complexes of argillite is leaching of metals into the cultivation medium that can be measured by atom absorption spectrometry (AAS) or ion coupled plasma spectrometry (ICP-MS). Among metals contained in argillite, Mo, Ni, Re, U, V, Co are in organometallic complexes.

A characteristic microbial community is the third evidence on degradation of organometallic complexes of argillite. This is determined by sampling the cultivation medium, centrifuging the sample to separate the microbial biomass, from which, in turn, the DNA is isolated and sequenced by the 16S rRNA gene, using mass-sequencing techniques (454 Life Sciences pyrosequencing, MySeq Illumina, etc.). Cultivation medium stimulating methane generation and metal leaching is dominated by the class Bacilli, also the members of genus Methanosarcina can be found. The class Clostridia, mainly genus Desulfotomaculum related to sulfur metabolism is dominating in cultivation media lacking methane generation. Equilibrium between sulfate reducers and methanogens is important to direct the process towards methanogenesis.

Shale bioleaching experiments performed worldwide have been, as a rule, conducted with access to oxygen—in this case “simple organic matter” (organic acids, aliphatic and aromatic hydrocarbons) stays in aerobic environment in the solution, where it can hinder the bioleaching of metals [Matlakowka et al., 2013]. However, with the method described in the present invention, in anaerobic environment with the aid of microorganisms methane gas is generated from the “simple organic matter”.

### DESCRIPTION OF THE PREFERRED EMBODIMENTS

On the method described in the invention decomposition of organometallic complexes of graptolite-argillite by a stable microbial consortium, accompanied by bioleaching of metals and methane gas release we provide the following evidence.

With the microbial medium R2A (1.5-3.0 g/L) used in the present invention supplemented with betaine (0.675 to 1.35 g/L) and using adapted microbial consortium as an inoculum, up to 7.92±0.39 liters of methane (354±17 μmol) per kg of argillite was released into the gas phase at a temperature of 37□° C. in anaerobic cultivation experiment in argon atmosphere (FIG. 1b). The biodegradable part of argillite organic matter amounted to 19.86±0.98% of the total organic matter. Adapted culture performed without lag-phase but equally well with the non-adapted culture (lag phase of up to 50 days) (FIG. 1a).

The origin of methane was verified by δ¹³C isotopic analysis method. The average values for δ¹³C (‰ V-PDB) for methane from the samples containing argillite and medium and from the samples without argillite, containing only medium (blank samples) were −51.99±4.60‰ and −72.86±5.35‰, correspondingly (FIG. 2). The typical δ¹³C value for methane originating from kerogen matter generated by aceticlastic pathway is known to be −50‰.

With the microbial medium R2A (3.0 g/L) used in the present invention, 26.2% cobalt and 9.14% nickel of the maximum concentration of these metals in argillite was bioleached into the growth medium under anaerobic conditions in argon atmosphere (FIG. 3). Both elements are necessary as cofactors of bacterial and archaeal enzymes.

The change of external characteristics of mineral is also an evidence on the decomposition of organometallic complexes in argillite. To be used in experiments, a drill core containing the mineral (∅10 cm, FIG. 5a) was crushed into pieces of 1-2 cm in size (FIG. 4). In the experiments with methane evolution, during cultivation the mineral was crumbled into sand-like material, forming blackish suspension 3 in the cultivation medium, where evolution of gas bubbles was noticeable (FIG. 5a). In cultivation experiments, wherein the evolution of methane was modest or nonexistent, the cultivation medium remained transparent (FIG. 5b), like the blank 2, which contained only the medium (FIG. 5a).

By the results of pyrosequencing, the medium stimulating methane generation R2A plus betaine was dominated by the class Bacilli—by bacterial 16S rRNA gene-specific primers the genus Ureibacillus, and by archaeal 16S rRNA gene-specific primers the family Bacillaceae, but also the methanogenic genus Methanosarcina (FIG. 6 and FIG. 7). Ni-enzyme urease containing genus Ureibacillus accounted for 87.43%, and Co, and Ni-enzymes containing genus Methanosarcina formed 3.69% of total taxa. By contrast, the cultivation media R2A and R2A plus methanol were dominated by representatives of the class Clostridia, mainly genus Desulfotomaculum related to sulfur metabolism, which accounted for 50-85% of all taxa assigned.

The microbial consortium described survives maintaining in growth medium with argillite at a temperature of 37□°C. up to four months and is suitable for stable inoculating of new cultures (in 1/20 scale) and for long-term storage as a stock culture at a temperature of −80□° C.

The features and advantages described herein are not all-inclusive and, looking at the drawings, detailed description, and claims, many additional features and advantages are apparent to ordinary skill. Furthermore, it should be noted that the language of the description has been principally selected for readability and instructional purposes, and not to limit the scope of invention.

### THE LIST OF DRAWINGS AND OTHER ILLUSTRATIVE MATERIAL

FIG. 1—Dynamics and yield of methane evolution from argillite using the growth medium R2A plus betaine: a) with indigenous microbial consortium of argillite, non-adapted to growth medium; b) with microbial consortium adapted to growth medium.

FIG. 2—Determination of the origin of the methane by isotopic analysis ((δ¹³C method).

FIG. 3—Bioleaching of metals from argillite in various growth media; Y-axis represents the yield of metal from its maximum concentration in argillite (enrichment value).

FIG. 4—Argillite sample prepared for cultivation experiment with particles dimensions of 1-2 cm.

FIG. 5—Change of external characteristics of argillite on cultivating in growth medium: a) in experiments with methane evolution a blackish suspension was formed; b) in experiments where methane evolution was modest or nonexistent, the growth medium remained transparent. 1—Section of argillite drill core, 2—reactor with growth medium and microbial consortium, 3—reactor with growth medium, argillite and microbial consortium.

FIG. 6—Species detected by pyrosequencing from the communities in various growth media with primer pair BSR357-BSF8 suitable for the bacterial 16S rRNA V2 region [McKenna, et al., 2008]: a) percentage of different taxa (operational taxonomic unit, OTU); b) the part of most important taxa in the community.

FIG. 7—Species detected by pyrosequencing from the communities in various growth media with primer pair Arch349F V2-A934B suitable for the archaeal 16S rRNA V2 region [Takai et al., 2000; Grosskopf et al., 1998]: a) percentage of different taxa (operational taxonomic unit, OTU); b) the part of most important taxa in the community.

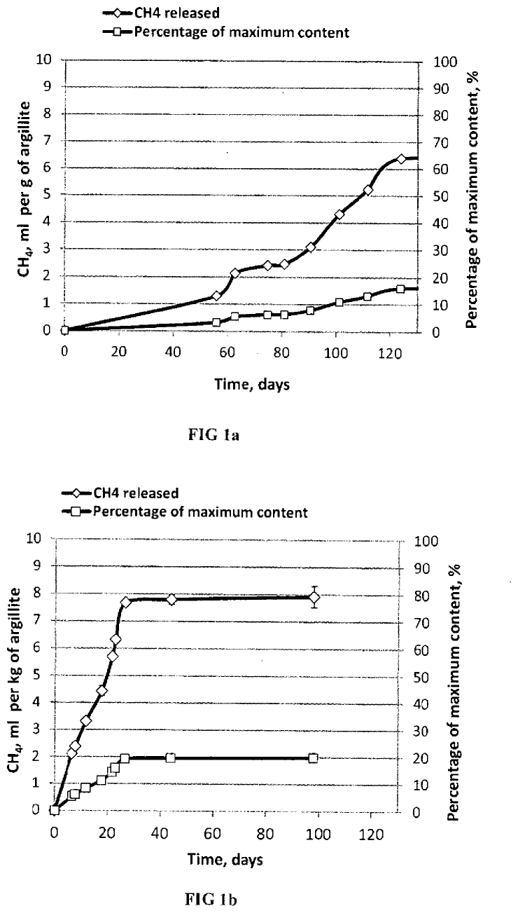

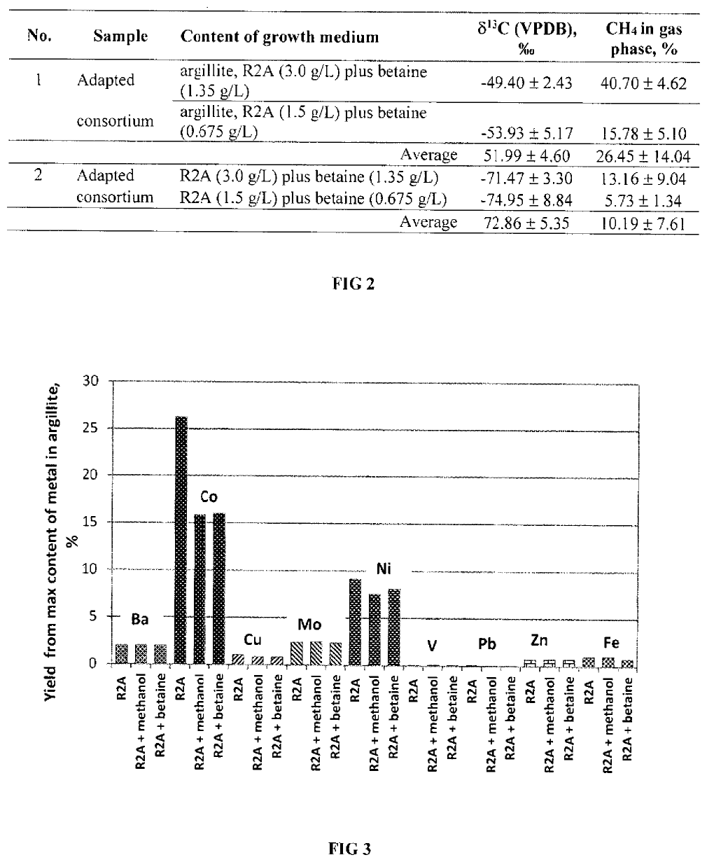

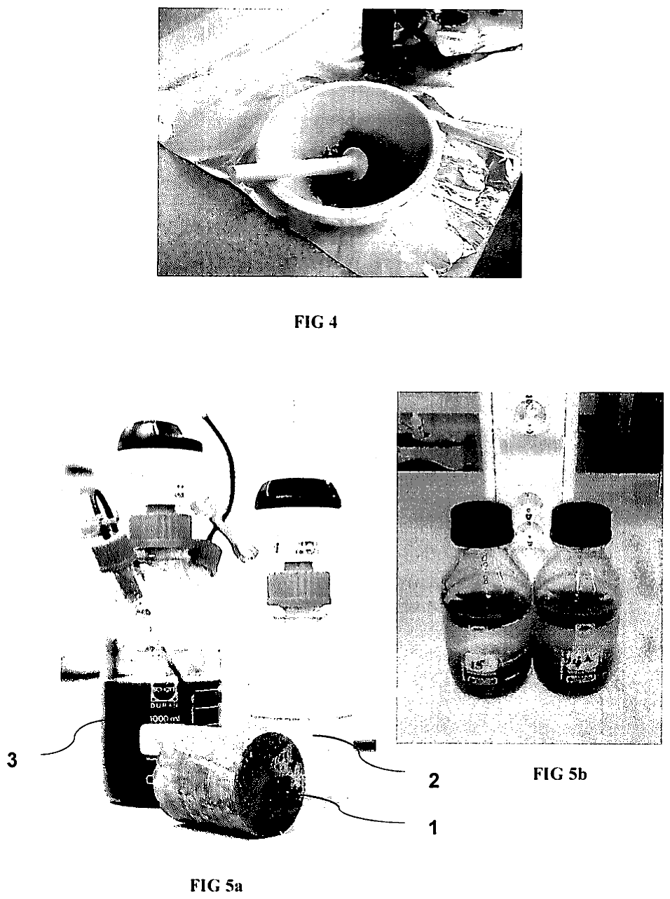

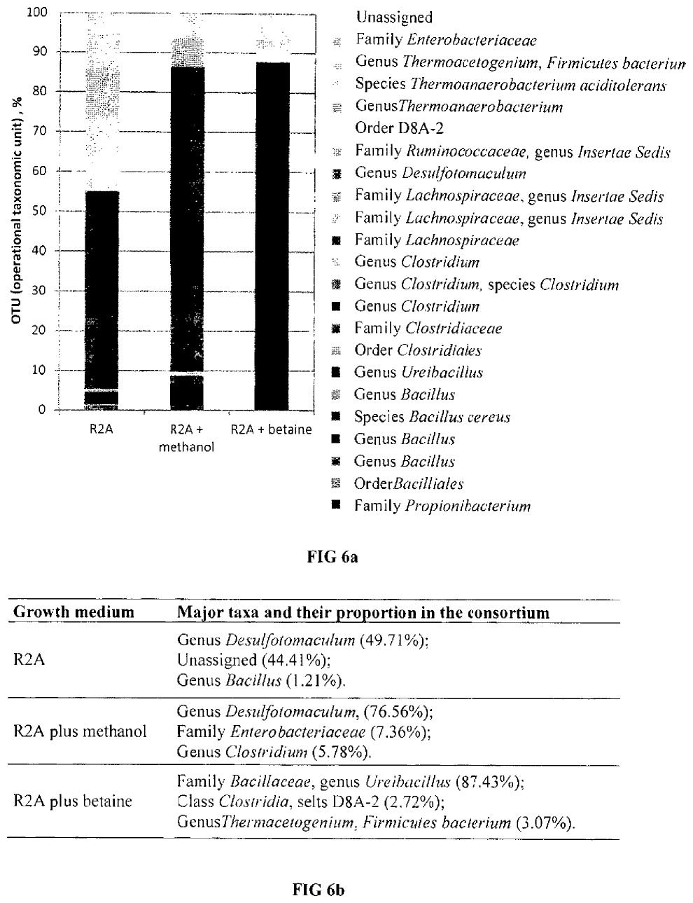

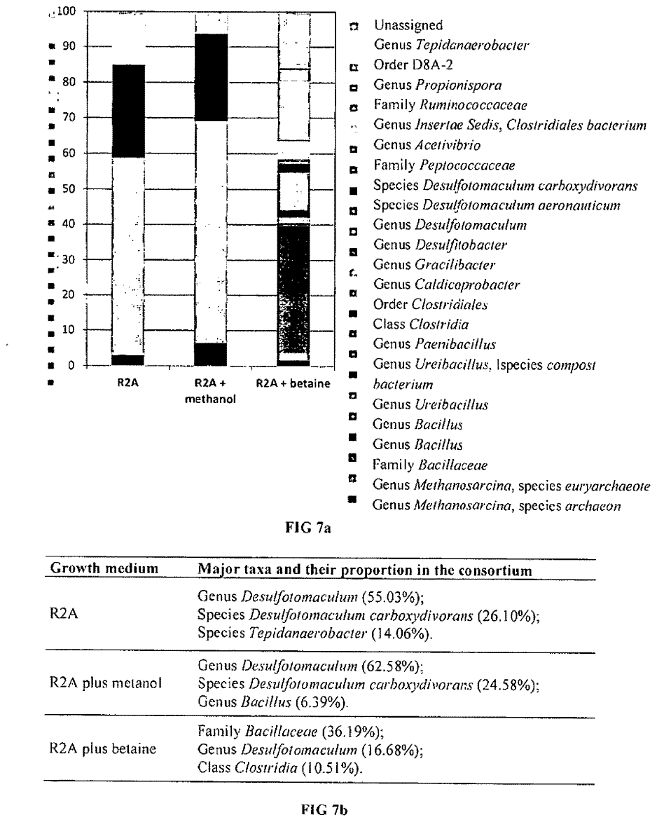

Example 1. With the method described in the invention, methane generation into the gas phase was initiated with an indigenous to argillite non-adapted consortium and medium R2A plus betaine in anaerobic cultivation experiment in argon atmosphere in a 500 mL test flask (FIG. 5) at a temperature of 37° C. and at pH 7.5. The gas phase pressure was measured by manometric system OxiTop (WTW, Germany), and the gas phase composition was analyzed with a gas chromatograph GC-2014 (Shimadzu, Japan; methane measurement range 10 ppb-30%). Using 25 g of crushed argillite as a substrate (with particle dimensions of 1-2 cm) within 90 days 417 ml of gas with methane content from 15 to 28%, with a yield of 3.1 liters of methane per 1 kg of argillite was obtained (FIG. 1a). 671 ml of biogenic gas with methane content of up to 37.5% was evolved as a maximum, which means a yield of 6.4 liters methane per 1 kg of mineral (argillite). On day 77 the culture media were sampled for liquid phase to determine the metal content by the flame-AAS method (ISO 8288). 26.2% cobalt and 9.14% of nickel of the maximum concentrations of these metals in the original sample had been leached into cultivation medium (FIG. 3). On the same day samples were taken from cultivation media for identification of microorganisms. Samples were centrifuged (5000 rev/min, 10 min) to separate the biomass of microorganisms, from which in turn the DNA was isolated with DNA Powersoil kit (MoBio, USA) and sequenced by 16S rRNA gene, using the pyrosequencing technology of 454 Life Sciences and primers according to the reference [Uuring Eesti argilliidist . . . , 2014]. In the growth medium R2A plus betaine with a primer pair BSR357-BSF8 suitable for amplification of bacterial 16S rRNA gene, bacterial genus Ureibacillus accounted for 87.43%, class Clostridia, order D8A-2 for 2.72%, and genus Thermacetogenium, Firmicutes bacterium for 3.07% of all taxa (FIG. 6). With a primer pair Arch349F-A934B suitable for amplification of archaeal 16S rRNA gene, archaeal genus Methanosarcina accounted for 3.69% and bacterial order Bacillacae for 36.25%, bacterial genus Desulfotomaculum for 16.7% and bacterial class Clostridia for 10.5% of all taxa identified (FIG. 7).

Example 2. Using freshly ground argillite and growth medium R2A plus betaine a new experiment was launched with a sample taken from the cultivation medium of Example 1 on day 129 (5% inoculum) in anaerobic conditions in argon atmosphere in a 1000 mL test flask 3 (FIG. 5a)) at a temperature of 37° C. and at pH 7.5. The gas phase pressure was measured by manometric system OxiTop (WTW, Germany); the gas phase composition was analyzed with gas chromatographs GC-2014 (Shimadzu, Japan; methane measurement range 10 ppb-30%) and Varian Inc., Model CP-4900 (methane measurement range 1-100%). Using 50 g of crushed argillite as a substrate (with particle dimensions of 1-2 cm) 7.92±0.39 liters of methane (354±17 μmol) per kg of argillite was evolved (FIG. 1b). Methane originated from the organic fraction of argillite, because the average values for δ¹³C (‰ V-PDB) for methane from the samples containing argillite and medium and from the samples without argillite, containing only medium (blank samples) were −51.99±4.60‰ and −72.86±5.35‰, correspondingly (FIG. 2). The biodegradable part of argillite organic matter amounted to 19.86±0.98% of the total organic matter. Thus with adapted microbial consortium and growth medium R2A plus betaine, using freshly ground argillite, 1.4 times more methane was obtained than has been previously extracted from similar black shales. Methane release from cultivation medium started immediately without a lag-phase (FIG. 1b), and argillite was disintegrated into fine suspension material 3 (FIG. 5a)

### REFERENCES

1. Aaloe, A.; Bauert, H.; Soesoo, A. Kukersiit-Eesti põlevkivi. MTÜ GEOGuide Baltoscandia, Tallinn. 2006.

2. Asakawa, S.; Sauer, K.; Liesack, W.; Thauer, R. K. (1998) Tetramethylammonium:coenzyme M methyltransferase system from Methanococcoides sp. Arch Microbiol, 170, 220-226.

3. Ashby, M; Wood, L.; Lidstrom, U.; Clarke, C.; Gould, A.; Strapoc, D.; Lambo, A. J.; Huizinga, B. J. (2013) Compositions and methods for identifying and modifying carbonaceous compositions Patent application US 20130116126 A1.

4. Bauert, H.; Kattai, V. (1997). Kukersite oil shale. Kogumikus A. Raukas & A. Teedumäe (Toim.). Geology and mineral resources of Estonia. Estonian Academy Publishers, Tallinn. 436 pp. ISBN 9985-50-185-3.

5. Clement, B. G.; Ferry, J. G.; Underwood, S. (2011) Methods to stimulate biogenic methane production from hydrocarbon-bearing formations. Patent application CA2801558 A1.

6. Grosskopf, R.; Janssen, P. H.; Liesack, W. (1997) Diversity and structure of the methanogenic community in anoxic rice paddy soil Microcosms as Examined by Cultivation and Direct 16S rRNA Gene Sequence Retrieval. Applied and Environmental Microbiology, 64, 960-969.

7. Harris, S. H.; Smith, R. L.; Barker, C. E. (2008) Microbial and chemical factors influencing methane production in laboratory incubations of low-rank subsurface coals. International Journal of Coal Geology, 76, 46-51.

8. Jones, E. J. P.; Voytek, M. A.; D. Warwick, P. D.; Margo D. Corum, M. D.; Cohn, A.; Bunnell, J. E.; Clark, A. C.; William H. Orem, W. A. (2008) Bioassay for estimating the biogenic methane-generating potential of coal samples. International Journal of Coal Geology, 76, 138-150.

9. Lippmaa, E. Maremäe, E., Pihlak, A.-T., Aguraiuja, R. (2009) Estonian graptolitic argillites—ancient ores or future fuels? Oil Shale, 26(4) 530-539.

10. Lippmaa, E., Maremäe, E., Pihlak, A.-T. (2011) Resources, production and processing of Baltoscandian multimetal black shales. Oil Shale, 28(1) 68-77.

11. Maremäe, E. (1988) Utilization of Estonian Alum Shale in the national economy. Oil Shale, 1988, 5(4), 407-417.

12. Matlakowka, R.; Ruszkowski, D.; Sklodowska, A. (2013) Microbial transformations of fossil organic matter of Kupferschiefer black shale—elements mobilization from metalloorganic compounds and metalloporphyrins by a community of indigenous microorganisms. Physicochemical Problems of Mineral Processing, 49 (1), 223-231.

13. McKenna, P.; Hoffmann, C.; Minkah, N.; Aye, P. P.; Lackner, A.; Liu, Z.; Lozupone, C. A.; Hamady, M., Knight, R.; Bushman, F. D. (2008). The macaque gut microbiome in health, lentiviral infection, and chronic enterocolitis. PLoS Pathogens 4(2), e20.

14. Meslé, M.; Periot, C.; Dromart, G.; Oger, P. (2013) Biostimulation to identify microbial communities involved in methane generation in shallow, kerogen-rich shales, Journal of Applied Microbiology, 114(1), 55-70, doi:10.1111/jam.12015.

15. Meslé, M.; Périot, C.; Gilles Dromart G.; Oger, P. (2015) Methanogenic microbial community of the Eastern Paris Basin: Potential for energy production from organic-rich shales. International Journal of Coal Geology, 149, 67-76.

16. Newell, C. J; Adamson, D. T.; Connor, J. A. (2008) Methods and systems for stimulating biogenic production of natural gas in the subsurface. Patent application WO2008/041990.

17. Petersell, V. Diktüoneemakilt, energia ja keskkond. Keskkonnatehnika, 2008, 8.

18. Pfeiffer, R. S.; Ulrich, G.; Vanzin, G.; Dannar, V.; Debruin, R. P.; Szaloczi, E. L. (2006) Methanogenesis stimulated by isolated an aerobic consortia. Patent WO 2006/118569 A1.

19. Pfeiffer, R. S.; Ulrich, G. A.; Finkelstein, M. (2010) Chemical amendments for the stimulation of biogenic gas generation in deposits of carbonaceous material. U.S. Pat. No. 7,696,132.

20. Pfeiffer, R. S.; Ulrich, G.: Vanzin, G.; Dannar, V.; Debruin, R. P.; DeBruyn, R. P.; Dodson, J. B. (2011) Biogenic fuel gas generation in geologic hydrocarbon deposits. U.S. Pat. No. 8,302,683.

21. Puura, E., Neretnieks, I., Kirsimäe, K. (1999) Atmospheric oxidation of the pyritic waste rock in Maardu, Estonia. 1. Field study and modelling. Environmental Geology 39 (1), 1-18.

22. Reinsalu, E. (2012). Fosforiit kui Eesti loodusvara. Eesti Loodus, 2012/3.

23. Sepp, H. (2013) Holotseeni paleokeskkonna muutused Loode-Eestis järvesetete stabiilsete isotoopide ja jälgelementide põhjal Turvaste Valgejärve läbilõikest. Magistritöö, Tartu Ülikool, 2013.

24. Sevinsky J. R.; Vanzin, G. F.; Haveman, S. A.; Kotler, N. R.; Mahaffey, W. (2015) Methods of stimulating acetoclastic methanogenesis in subterranean deposits of carbonaceous material. U.S. Pat. No. 9004162 B2

25. Takai, K.; Horikoshi, K. (2000) Rapid detection and quantification of members of the archaeal community by quantitative PCR using fluorogenic probes. Applied and Environmental Microbiology, 66, 5066-5072.

26. Ticak, T.; Hariraju, D.; Bayron Arcelay, M.; Arivett, B. A.; Fiester, S. E.; Ferguson Jr, D. J. (2015) Isolation and characterization of a tetramethylammonium degrading Methanococcoides strain and a novel glycine betaine utilizing Methanolobus strain. Archives of Microbiology, 197(2), 197-209.

27. Toledo, G. V.; Richardson, T. H.; Stingl, U.; Mathur, E. J., Venter, J. C. (2009) Methods to stimulate biogenic methane production from hydrocarbon-bearing formations. Patent application WO2009/140313.

28. Urios, L.; Marsal, F.; Pellegrini, D.; Magot, M. (2012) Microbial diversity of the 180 million-year-old Toarcian argillite from Tournemire, France. Applied Geochemistry, 27 (7), 1442-1450, doi: 10.1016/j.apgeochem.2011.09.022.

29. Urios, L.; Marsal, F.; Pellegrini, D.; Magot, M. (2013) Microbial diversity at iron-clay interfaces after 10 years of interaction inside a deep argillite geological formation (Tournemire, France), Geomicrobiology Journal, 30:5, 442-453, doi: 10.1080/01490451.2012.705227.

30. Uuring Eesti argilliidist biogeense metaangaasi puuraugus (in situ) tootmise võimalikkuse tõestamiseks. Lõppraport. Vastavalt lepingule nr 4.3_2.14.420, sõlmitud 8.03.14 Ettevõtluse Arendamise Sihtasutuse ja BiotaP oÜ vahel. Tallinn, 2014. 22.08.2014 http://www.eas.ee/images/doc/sihtasutusest/uuringud/ettevotlus/uuring-argilliidist-biogeense-metaangaasi.pdf (külastatud 20.10.2016)

31.Voolma, M.; Soesoo, A., Hade, S., Hints, R., Kallaste, T. (2013) Geochemical heterogeneity of Estonian graptolite argillite. Oil Shale, 30(3) 377-401.

32. Watkins, A. J.; Roussel, E. G.; R. Parkes, R. J. Sass, H. (2014) Glycine betaine as a direct substrate for methanogens (Methanococcoides spp.) Applied and Environmental Microbiology. 80 (1), 289-293.

33. Wuchter, C.; Banning, E.; Mincer, T. J.; Drenzek, N. J.; Coolen, M. J. L. (2013) Microbial diversity and methanogenic activity of Antrim Shale formation waters from recently fractured wells. Frontiers in Microbiology, 4, 367, doi: 10.3389/fmicb.2013.00367.

1. A ethod for decomposition of organometallic matter of graptolite-argillite by a microbial consortium which leads to release of biogenic methane, the method comprising a step of using a liquid growth medium R2A plus betaine for producing methane, and bioleaching of metals occurs in an anaerobic medium.

2. The method according to claim 1, wherein the metals leached are nickel and cobalt.

3. The method according to claim 1, wherein for the release of biogenic methane from the organometallic material, accompanied by metal leaching a microbial community inherent to argillite is used.

4. The method according to claim 3, wherein when inoculating fresh argillite samples with microbial community inherent to argillite, a new adapted consortium with better biodegrading capability will be achieved that gives a higher methane yield.
